(19) United States
(12) Patent Application Publication
(10) Pub. No.: US 2022/0023333 A1
(43) Pub. Date: Jan. 27, 2022

(54) METHOD FOR INCREASING CANCER PATIENT'S HAEMOGLOBIN LEVEL

(71) Applicants: Elena Valentinovna ARSHINTSEVA, Saransk (RU); Sergei Yurevich PUSHKIN, Noginsk-9 (RU)

(72) Inventors: Elena Valentinovna ARSHINTSEVA, Saransk (RU); Sergei Yurevich PUSHKIN, Noginsk-9 (RU)

(21) Appl. No.: 17/312,022

(22) PCT Filed: May 6, 2019
(86) PCT No.: PCT/RU2019/000307

§ 371 Date: Jun. 9, 2021

(51) Int. Cl. A61K 31/77; A61P 7/06

(52) U.S. Cl. CPC A61K 31/77; A61P 7/06

(57) ABSTRACT

The invention relates to the field of medicine, particularly to a method for increasing blood haemoglobin level in a cancer patient. A method for increasing blood haemoglobin level in a cancer patient, wherein a pharmaceutical composition comprising a poloxamer as a pharmacologically active substance is administered before and/or during and/or after a course of the chemotherapy or immunotherapy is provided. Thus, the claimed invention provides such a method for increasing blood haemoglobin level in a cancer patient, implementation of which ensures the achievement of the technical result consisting in normalization and maintaining patient's general physical condition at a level that allows for chemotherapy and immunotherapy courses to be administered and improves patients general physical condition following the treatment of cancer with simultaneous reduction of a toxic effect and side effects on a patients

The invention relates to the field of medicine, particularly to a method for increasing blood haemoglobin level in a cancer patient by administering to the patient a pharmaceutical composition of a poloxamer before and/or during and/or after a course of the chemotherapy or immunotherapy.

The reduced blood haemoglobin level in a cancer patient can be caused both by the presence of a tumor and by the treatment of the disease itself, for example, as a result of the chemotherapy or immunotherapy. Generally, anaemia is a condition characterized by a decrease in haemoglobin content (up to 115-110 g/l and below) per unit of blood volume, leading to a failure in tissue oxygen supply ( [symbol] A. B., [symbol] [symbol] y [symbol] : [symbol] [symbol] .— [symbol] : [symbol] , 2008.—384 c. (Papayan A. V., Zhukova L. Yu. Anemii u detey: rukovodstvo dlya vrachey.—St. P.: Piter, 2001.—384 p.)). The presence of anaemia in oncological patients adversely affects their general physical condition, which is generally characterized by a weakness in a patient, which consequently has an impact on quality and duration of patient's life. Chemotherapy currently occupies one of the leading positions in the treatment of cancer patients, despite the presence of a variety of adverse events associated with systemic toxicity, limited efficiency and serious side effects, and anaemia caused, inter alia, by a cancer itself is the main contraindication for a prescription of the chemotherapy. Another alternative treatment option for cancerous diseases is immunotherapy, the toxic effects of which on a patient's body are minimized compared to chemotherapy. In some cases, during the treatment of malignant diseases immunotherapy may exhibit hematological toxicity, thereby inducing anaemia.

Thus, it is reasonable to provide a treatment associated with increase in blood haemoglobin level or prevention of blood haemoglobin level reduction in a patient suffering from cancer, in order to prevent the development of anaemia, improve patient's general physical condition, as well as to ensure further possibility for administration of the chemotherapy or immunotherapy.

For the treatment of anaemia in cancer patients, donated red blood cells or erythropoietin are generally used in order to reduce a need for donor blood transfusion [symbol] [symbol] [symbol] [symbol] [symbol] [symbol] / [symbol] B. B. [symbol] , A. A. [symbol] // [symbol] [symbol] [symbol] .—2017.—T.4.— [symbol] 2.—C. 37-42 (Berezin G. P. Rol' eritropoetinov v lechenii anemii u onkologicheskikh bol'nykh/P. G. Berezin, V. V. Milovanov, A. A. Ivannikov//Issledovaniya i praktika v meditsine. 2017.—V.4.— [symbol] 2. P.—37-42)). Donor blood transfusion may be accompanied by post-transfusion complications, and this method is practically not used in outpatient settings. Thus, a more acceptable method is the use of recombinant erythropoietin, which stimulates proliferation of erythroid lineage in the bone marrow, leading to an increase in blood haemoglobin content in oncological patients, thereby improving quality and duration of their life. According to the data provided in the above article, following a therapy with erythropoietin drug products more than 81% of oncological patients had an increase in haemoglobin level.

However, the use of erythropoietin may be accompanied by a number of side effects, as described in the document RU2642302, published on 24.01.18. Recently disclosed information suggests that the use of higher doses of erythropoietin may be associated with an increased risk of cardiovascular diseases, tumor growth and mortality in some patient populations (Kraft et al., 2009, Clin J Am Soc Nephrol 4:470-480; Glaspy, 2009, Annu Rev Med 60:181-192). Also, erythropoietin does not always show efficacy, and many patients are resistant to high doses of the said active substance (Horl et al. (2000) Nephrol Dial Transplant 15, 43-50). In addition, more than 50% of patients suffering from malignant diseases have an inadequate response to erythropoietin. About 10% of patients with severe kidney disease have a decreased response to erythropoietin (Glaspy et al. (1997) J Clin Oncol 15, 1218-1234; Demetri et al. (1998) J Clin Oncol 16, 3412-3425). Only less than 10% of patients with myelodysplastic syndrome are known to respond favorably to treatment with erythropoietin (Estey (2003) Curr Opin Hematol 10, 60-67).

Thus, disadvantages of erythropoietin usage may include insufficient effectiveness in a number of the above cases, as well as arising of adverse effects as a result of receiving higher doses of erythropoietin. It should also be noted that the cultivation of erythropoietin is a quite time-consuming and complex process.

The problem addressed by the present invention is to provide a method for increasing blood haemoglobin level in a cancer patient, implementation of which would ensure the achievement of the technical result consisting in normalization and maintaining patient's general physical condition at a level that allows for chemotherapy and immunotherapy courses to be administered and improves patient's general physical condition following the treatment of cancer with simultaneous reduction of a toxic effect and side effects on a patient's body.

The problem set forth is solved by providing a method for increasing blood haemoglobin level in a cancer patient, wherein a pharmaceutical composition comprising a poloxamer as a pharmacologically active substance is administered before and/or during and/or after a course of the chemotherapy or immunotherapy.

It is known that poloxamers are polyoxyethylene and polyoxypropylene block copolymers, which are used not only as excipients, but also as drug substances that have useful biological properties.

Poloxamer has the most widespread use as an emulsifier for perfluoroorganic compound based emulsions for intravenous administration, as well as a stabilizer that provides transparency to elixirs and syrups. In addition, poloxamer is used as a wetting agent in eye drops, ointments, gels and as a binding agent in tablets. Poloxamers are also used for the treatment of pathological hydrophobic interactions in the blood and in other biological fluids, since they improve blood flow and reduce the adhesion of macromolecules and cells.

Commercial poloxamers are known under the following trade names: Proxanol, Emuxol, Kolliphor, Pluronic, Synperonic, Lutrol.

In view of poloxamer specific properties that define their use in medicine, the inventors suggested a possibility of using a pharmaceutical composition of a poloxamer to prevent further development of anaemia resulting from oncological disease or treatment thereof and to improve patient's general condition as a result of increasing blood haemoglobin content to the acceptable level, thereby allowing for easy administration of the chemotherapy or immunotherapy. Furthermore, it precludes the use of the recombinant erythropoietin, which prevents adverse events caused by the use of the aforementioned hormone. There have been found out the properties of poloxamer to stimulate the erythrocyte lineage of the bone marrow, thus ensuring the achievement of said purposes. In addition, it is known that poloxamers are low toxic substances and do not have side effects intrinsic to erythropoietin when administered to a patient body.

The term “cancer patient”, as used herein, refers to human diagnosed with at least one of the following cancerous diseases: prostate cancer, colorectal cancer, pancreatic cancer, cervical cancer, gastric cancer, endometrial cancer, brain cancer, liver cancer, bladder cancer, ovary cancer, testicular cancer, head and neck cancer, skin cancer (including melanoma and basal carcinoma), pleural mesothelioma, lymphocytic cancer (including lymphoma and leukaemia), esophageal cancer, breast cancer, muscle tissue cancer, connective tissue cancer, lung cancer (including small cell carcinoma and non-small cell carcinoma), renal cell carcinoma, gastric carcinoma, sarcoma, choriocarcinoma, basal cell skin carcinoma, squamous cell skin carcinoma and ovarian cancer, which are not, however, limited to the given examples.

The term “chemotherapy”, as used herein, refers to the treatment of malignant neoplasms with chemotherapeutic preparations that inhibit proliferation of malignantly transformed cells of the body or irreversibly damage these cells.

The term “immunotherapy”, as used herein, refers to the treatment of malignant neoplasms with preparations that enhance or restore the function of the immune system.

According to the present invention, during the chemo- or immunotherapy the following active agents are used: antibodies or fragments thereof (for example, nivolumab, trastuzumab, bevacizumab, rituximab, panitumumab), antimetabolites (for example, methotrexate, cytarabine, gemcitabine, capecitabine), DNA-damaging preparations (for example, mitoxantrone, doxorubicin, carminomycine, nitrosourea, thiotepa, altretamine, platinum-based agents and combinations thereof), tubulin-interactive preparations (for example, paclitaxel, docetaxel, vinorelbine), hormonal preparations (for example, prednisolone, letrozole, tamoxifen, flutamide), antimitotic preparations (navelbine, paclitaxel, vinblastine, vinorelbine), antiangiogenic preparations (for example, avastin, vadimizan, thalidomide), which are not, however, limited to the given examples.

A pharmaceutical composition according to the present invention comprises at least one poloxamer together with one or more pharmacologically acceptable carriers and optionally other therapeutic ingredients. The carrier(s) must be “acceptable” in the sense of being compatible with the other ingredients of the formulation and physiologically innocuous to the recipient.

It is preferable to administer the pharmaceutical composition in the form of a tablet, effervescent tablet, capsule, powder, solution or emulsion.

One of the acceptable solid forms of the pharmaceutical composition of the present invention are tablets containing an active ingredient in a mixture with non-toxic pharmacologically acceptable excipients that are suitable for the tablet manufacturing. These excipients may be, for example, inert diluents, such as calcium or sodium carbonate, lactose, lactose monohydrate, croscarmellose sodium, povidone, calcium or sodium phosphate; granulating and disintegrating agents such as corn starch or alginic acid; binders such as cellulose, microcrystalline cellulose, starch, gelatin, or acacia; and lubricants such as magnesium stearate, stearic acid or talc.

Other acceptable solid form of the pharmaceutical composition of the present invention that is suitable for oral administration includes hard gelatin capsules, in which an active ingredient is mixed with an inert solid diluent, or soft gelatin capsules, in which an active ingredient is mixed with an aqueous or oil medium.

Formulations of solutions according to the present invention that are suitable for oral and parenteral administration may be presented in the form of aqueous solutions or emulsions of perfluoroorganic compounds, wherein water for injection or purified water are preferred solvents.

Parenterally administered pharmaceutical compositions according to the present invention may be presented in the form of a sterile composition for injection, such as a sterile aqueous solution. Additionally, in the aqueous formulations may be included metal salts such as sodium chloride, potassium chloride, magnesium chloride, as well as the following additional components: glucose, ascorbic acid, inosine. In this case the quantitative content of additional components in the said composition is: for sodium chloride—0.3-0.9 wt %, for potassium chloride—0.03-0.04 wt %, for magnesium chloride—0.01-0.02 wt %, for glucose—0.5-10 wt %, for ascorbic acid—0.1-1.0 wt %, and for inosine—0.1-1.0 wt %.

Formulations of compositions intended for oral and parenteral administration in the form of emulsions of perfluoroorganic compounds with the concentration of a poloxamer in the range of 1-16 wt %/1 comprise perfluorodecalin and perfluoromethylcyclohexylpiperidine in concentrations of 0.001-60 wt %/l, emulsifying and stabilizing additive, and may further comprise sodium chloride, potassium chloride, magnesium chloride, sodium hydrogen carbonate, sodium phosphate monobasic and glucose.

Solutions and emulsions comprising a poloxamer in their composition can be packaged in containers, such as ampoules or vials.

Formulations of pharmaceutical compositions according to the present invention can be prepared by any method known in the pharmaceutical art.

For example, tablets, capsules, powders are manufactured according to known techniques for solid dosage form production, for example, by wet granulation method followed by addition of a lubricant to the dry granules, molding the final mixture of ingredients to form a dosage form of a given configuration and size, and coating, if necessary.

Solutions for oral and parenteral administration are prepared by the use of an appropriate solvent, and in the case of solutions for injections following all standards for aseptic technique.

In turn, to obtain, for example, emulsions of perfluoroorganic compounds, a common method is used, which is based on the techniques that utilize ultrasound or homogenization on disintegrators under high pressure.

Administration of the pharmaceutical composition comprising a poloxamer before and/or after a course of the chemotherapy and immunotherapy is performed to increase the blood haemoglobin level in a patient having haemoglobin levels, which are below normal, in order to ensure further possibility for administration of the chemotherapy or immunotherapy or in order to normalize patient's general physical condition. Administration of the said pharmaceutical composition is prescribed 1-7 days before the start of chemotherapy or immunotherapy course, it is also preferable to administer the pharmaceutical composition 8-14 days before the start of chemotherapy or immunotherapy course, and it is most preferable to administer the pharmaceutical composition 15-21 days before the start of chemotherapy or immunotherapy course.

In order to maintain the blood haemoglobin level in a cancer patient at an acceptable level, the pharmaceutical composition of a poloxamer is administered during the entire course of the chemotherapy or immunotherapy.

In a preferred embodiment of the present invention, a multiple oral or parenteral administration of the said pharmaceutical composition to a patient is performed.

In another preferred embodiment of the present invention, a poloxamer with the concentration of 1-28 wt % is used in a formulation of the pharmaceutical composition.

In yet another preferred embodiment of the present invention, a poloxamer-188 is used in a formulation of the pharmaceutical composition.

Below are implementation examples of the method for increasing blood haemoglobin level in a cancer patient by administering the pharmaceutical composition comprising a poloxamer as a pharmacologically active substance, wherein such trademarks of poloxamer-188 as Lutrol F68 (BASF), Emuxol-268 of Grade “A” and Kolliphor P188 were used.

Example 1. Increase in the haemoglobin level during the treatment of multiple myeloma by oral administration of the pharmaceutical composition comprising 13.3 wt % poloxamer before and during a course of the chemotherapy.

Patient G., man, 70 years of age. Diagnosis: progressive diffuse-nodular multiple myeloma of the stage IIIB. Myelogenic nephropathy, stage III CKD, terminal stage. Chronic programmed dialysis. The initial level of haemoglobin was 72 g/l. The receiving of EPO 20000 IU and iron preparations was prescribed for a month, as a result of which the level of haemoglobin increased to 84 g/l. However, the patient's condition worsened, and there were diagnosed acute transmural myocardial infarction and weight loss in the patient. The patient was prescribed the administration of 30 ml of the sterile pyrogen-free aqueous solution of the poloxamer comprising 13.3 wt % poloxamer-188 (Emuxol-268 of Grade “A”) and water for injection as the remaining part for 30 days. The level of haemoglobin was 116 g/l. Then, 30 ml of the said poloxamer solution was administered to the patient on a daily basis during 9 courses of chemotherapy (cyclophosphan and boramilane), while EPO and iron preparations were no longer prescribed. The level of haemoglobin was maintained in the range of 116-125 g/l.

Example 2. Increase in the haemoglobin level during the treatment of locally advanced or metastatic bladder cancer of the 4-^{th} stage with lumbar vertebral metastases by oral administration of the pharmaceutical composition comprising 4 wt % poloxamer during a course of the immunotherapy and after a course of the immunotherapy.

Patient A., man, 66 years of age. Diagnosis: locally advanced or metastatic bladder cancer of the 4t^{h} stage with lumbar vertebral metastases. The initial level of haemoglobin was 129 g/l.

Immunotherapy 1: the patient underwent a course of the therapy with the nivolumab preparation (Opdivo) for 4 months. Donated red blood cells were regularly transfused to increase the level of haemoglobin. After performed therapy, the level of haemoglobin dropped to 123 g/l. Said immunotherapy 1 resulted in a slight progress of treatment.

Immunotherapy 2: the patient underwent a course of the therapy with the nivolumab preparation (Opdivo) for 5 months with the daily administration of 50 ml of the sterile pyrogen-free aqueous solution of a poloxamer comprising 4 wt % poloxamer-188 (Emuxol-268 of Grade “A”) and water for injection as the remaining part. Following administration of the said pharmaceutical composition of a poloxamer, the level of haemoglobin increased from 123 g/l to 136 g/l. As a result of immunotherapy 2 patient's general condition improved significantly, and there were no anxiety symptoms. Administration of immunotherapy during the administration of the pharmaceutical composition comprising a poloxamer resulted in a better response to immunotherapy, and, which is also important, the immunotherapy proceeded without toxic complications.

Example 3. Increase in the haemoglobin level during the treatment of chronic lymphocytic leukaemia by oral administration of an emulsion of perfluoroorganic compounds comprising 4.0 wt % poloxamer before a course of the chemotherapy.

Patient B., man, 71 years of age. Diagnosis: chronic lymphocytic leukaemia. The initial level of haemoglobin was 92.0 g/l. The chemotherapy was not prescribed due to the low haemoglobin value. Iron preparations did not lead to an increase in haemoglobin level. 15 ml of the perfluoroorganic compound based emulsion comprising 4.0 wt % poloxamer-188 (Emuxol-268 of Grade “A”), 13.5 wt % perfluorodecalin, 6.5 wt % perfluoromethylcyclohexylpiperidine, 0.6 wt % sodium chloride, 0.09 wt % potassium chloride and water for injection as the remaining part was administered to the patient during 21 days without the use of other standard treatment preparations. The level of haemoglobin was 101 g/l. Based on the result of anemia treatment, a course of the chemotherapy with bendamustine was carried out.

Example 4. Increase in the haemoglobin level during the treatment of chronic lymphocytic leukaemia by intravenous administration of the pharmaceutical composition comprising 4 wt % poloxamer during a course of the chemotherapy.

Patient C., woman, 59 years of age. Diagnosis: chronic lymphocytic leukaemia. The initial level of haemoglobin was 118.0 g/l. For the prophylaxis of anaemia caused by chemotherapy, 10 ml of the sterile pyrogen-free aqueous solution of a poloxamer comprising 4 wt % poloxamer-188 (Kolliphor P188), 0.6 wt % sodium chloride and water for injection as the remaining part was administered to the patient simultaneously with a course of the bendamustine chemotherapy. The level of haemoglobin was 122.0 g/l. The patient underwent a course of the chemotherapy without complications.

Example 5. Increase in the haemoglobin level during the treatment of right kidney tumor with metastases by intravenous administration of the pharmaceutical composition comprising 6 wt % poloxamer after a course of the immunotherapy.

Patient D., woman, 58 years of age. Diagnosis: right kidney tumor with metastases T2N1M1. Coronary heart disease. The initial level of haemoglobin was 148 g/1. A course of the immunotherapy with the nivolumab preparation (Opdivo) was prescribed. After 1 course, the level of haemoglobin reduced to 104 g/l. For the treatment of immunotherapy induced anaemia, 20 ml of the pyrogen-free aqueous solution of a poloxamer comprising 6 wt % poloxamer-188 (Emuxol-268 of Grade “A”) and water for injection as the remaining part was administered to the patient during 90 days without the use of other standard treatment preparations. The level of haemoglobin was 130 g/l. Exacted diagnosis: right kidney tumor T1N1M0.

Example 6. Increase in the haemoglobin level during the treatment of pancreatic cancer IIB by intravenous administration of the pharmaceutical composition comprising 6 wt % poloxamer during a course of the chemotherapy.

Patient E., man, 61 years of age. Diagnosis: non-metastatic pancreatic cancer IIB. The initial level of haemoglobin was 104 g/l. A course of the chemotherapy with gemcitabine preparation was prescribed. During the entire course of the chemotherapy the patient was administered with 20 ml of the sterile pyrogen-free aqueous solution of a poloxamer comprising 6 wt % poloxamer-188 (Kolliphor P188) and water for injection as the remaining part. The level of haemoglobin was 112 g/l. After receiving of the above pharmaceutical composition during a course of the chemotherapy for 3 months, the pancreatic injury was not observed.

Example 7. Increase in the haemoglobin level during the treatment of lymphocytic leukaemia by oral administration of the pharmaceutical composition comprising 13.3 wt % poloxamer before, during or after a course of the chemotherapy.

Patient F., man, 65 years of age. Diagnosis: lymphocytic leukaemia. The initial level of haemoglobin was 104 g/l. The chemotherapy was not prescribed due to the low blood haemoglobin level in the patient. Receiving of iron preparations did not lead to an increase in haemoglobin level. 15 ml of the sterile pyrogen-free aqueous solution of a poloxamer comprising 13.3 wt % poloxamer-188 (Lutrol F 68) and water for injection as the remaining part was administered to the patient during 14 days without the use of other standard treatment preparations. After receiving of the above pharmaceutical composition, the level of haemoglobin was 113 g/l. Based on the result of anemia treatment, a course of the chemotherapy with bendamustine preparation was carried out with daily administration of 15 ml of the sterile pyrogen-free aqueous solution of a poloxamer comprising 13.3 wt % poloxamer-188 (Lutrol F 68) and water for injection as the remaining part. After a course of the chemotherapy with daily administration of the poloxamer preparation, the level of haemoglobin was 114 g/l. For further increasing of haemoglobin level, the above aqueous solution of the poloxamer was administered to the patient during 60 days. As a result the level of haemoglobin increased to 129 g/l.

Example 8. Increase in the haemoglobin level during the treatment of metastatic pancreatic cancer with liver metastases by oral administration of the pharmaceutical composition comprising 16.6 wt % poloxamer during and after a course of the chemotherapy.

Patient H., woman, 57 years of age. Diagnosis: metastatic pancreatic cancer with liver metastases. The initial level of haemoglobin was 107 g/l. A course of the chemotherapy with gemcitabine and abraxane was prescribed, and 10 ml of the sterile pyrogen-free aqueous solution of a poloxamer comprising 16.6 wt % poloxamer-188 (Emuxol-268 of Grade “N”) and water for injection as the remaining part was administered to the patient for 30 days. Following the use of the above pharmaceutical composition, the level of haemoglobin was 112 g/l. As a result of receiving the above pharmaceutical composition during a course of the chemotherapy there was no pancreatic injury, while significant regression of liver metastases was observed. For further increasing of haemoglobin level, 10 ml of the sterile pyrogen-free aqueous solution of the poloxamer comprising 16,6 wt % poloxamer-188 (Emuxol-268 of Grade “A”) and water for injection as the remaining part was administered to the patient for 60 days. As a result the level of haemoglobin increased to 132 g/l.

Example 9. Increase in the haemoglobin level during the treatment of third stage cervical cancer by oral administration of the pharmaceutical composition comprising 28 wt. % poloxamer during a course of the chemotherapy.

Patient I., woman, 74 years of age. Diagnosis: third stage cervical cancer. Coronary heart disease. The initial level of haemoglobin was 91 g/l. For the prophylaxis of anaemia caused by chemotherapy course, 10 ml of the sterile pyrogen-free aqueous solution of a poloxamer comprising 28 wt % poloxamer-188 (Kolliphor P188), 0.6 wt % sodium chloride and water for injection as the remaining part was administered to the patient during the entire course of the chemotherapy (paclitaxel). The level of haemoglobin following chemotherapy was 110 g/l.

The study conducted revealed that the level of blood haemoglobin in human have increased following intravenous and oral administration of pharmaceutical compositions comprising a poloxamer, furthermore, the inventors did not find any side effects due to receiving of said compositions.

The present invention is illustrated by the above examples, which are not intended to limit in any way the subject matter of the present invention. Course duration for administration of the pharmaceutical composition comprising a poloxamer according to the present method is determined by a physician and may encompass a quite wide time range. The minimum course for administration of the said composition is 14 days, and the maximum course for administration of the above-described pharmaceutical composition may last during the entire course of the chemotherapy or immunotherapy, and each unit dose may comprise a poloxamer in the range of 500-5000 mg.

Thus, the claimed invention provides such a method for increasing blood haemoglobin level in a cancer patient, implementation of which ensures the achievement of the technical result consisting in normalization and maintaining patient's general physical condition at a level that allows for chemotherapy and immunotherapy courses to be administered and improves patient's general physical condition following the treatment of cancer with simultaneous reduction of a toxic effect and side effects on a patient's body.

1. A method for increasing blood haemoglobin level in a cancer patient, wherein a pharmaceutical composition comprising a poloxamer as a pharmacologically active substance is administered before and/or during and/or after a course of the chemotherapy or immunotherapy.

2. The method according to claim 1, wherein the pharmaceutical composition in the form of a tablet, effervescent tablet, capsule, powder, solution or emulsion is administered.

3. The method according to claim 1, wherein a multiple intravenous or oral administration of the pharmaceutical composition is performed.

4. The method according to claim 1, wherein a poloxamer with the concentration of 1-28 wt % is used in a formulation of the pharmaceutical composition.

5. The method according to claim 1, wherein a poloxamer-188 is used in a formulation of the pharmaceutical composition.
